(19) United States
(12) Patent Application Publication
(10) Pub. No.: US 2021/0012902 A1
(43) Pub. Date: Jan. 14, 2021

(54) REPRESENTATION LEARNING FOR WEARABLE-SENSOR TIME SERIES DATA

(71) Applicant: University of Notre Dame du Lac, South Bend, IN (US)

(72) Inventors: Nitesh V. Chawla, South Bend, IN (US); Xian Wu, Notre Dame, IN (US)

(21) Appl. No.: 16/845,995

(22) Filed: Apr. 10, 2020

Related U.S. Application Data
(60) Provisional application No. 62/807,068, filed on Feb. 18, 2019.

(51) Int. Cl. G16H 50/30; G16H 50/20; G06K 9/62; G06F 3/01

(52) U.S. Cl. CPC G16H 50/30; G06F 3/011; G06K 9/628; G16H 50/20

(57) ABSTRACT

Presented herein are embodiments for analysis of and representation learning for wearable sensor time series data. Such wearable sensor time series data may come, for example, from wearable electronic heart rate sensors and monitors. Embodiments described herein may include systems, methods, and computer program products for analyzing time series data generated by or collected by a wearable sensor. Time series data may be variable length and may be incomplete.

### CROSS-REFERENCE TO RELATED APPLICATIONS

This application claims priority to and benefit from U.S. Provisional 62/807,068, titled “Machine Capable of Performing Deep Representation Learning for Temporal and Time Series Data,” filed on Feb. 18, 2019, which is incorporated herein by reference in its entirety.

### BACKGROUND

The prevalence of wearable sensors (e.g., smart wristbands, heart rate monitors, etc.) is enabling an unprecedented opportunity to inform health and wellness states of individuals, providing deeper personalized insight that goes beyond how many steps a user may take or a record of heart rate. However, before this goal of personalized insights for quantified users may be acheived, there are a number of challenges to be resolved regarding modeling continuous temporal data. For example, with heart rate, derived from wearable sensors:
1) data is of variable-length and is incomplete due to different data collection periods (e.g., wearing behavior varies by person);
2) there is intra-sensor and intra-individual variability;
3) inter-individual variability; and
4) lack of substantial ground truth.

The wide proliferation of wearable sensors and mobile devices is revolutionizing health and wellness with the potential of data and personalized insights at one's fingertips. These wearables generate chronologically ordered streams (e.g., the series of heart rate measurements) or point-in-time activity (e.g., the number of steps) or general summarization of the day (e.g., move or step goals). Collectively, these data provide an unprecedented opportunity to learn about health and wellness states, as well as how those interact with the social network, opinions, beliefs, personality, and job performance. Embodiments of the invention as described herein specifically focus on chronologically ordered data (such as the heart rate data), which is refered to herein as wearable-sensor time series data. It is important to be able to effectively model these these wearable-sensor time series data to fully realize their benefit for a wide spectrum of applications, such as personality detection, job performance prediction, health and wellness state assessment and prediction, user identification, demographics inference, and may other applications. However, wearable-sensory time series data come with a number of challenges, including temporal dependencies, incompleteness, intra-sensor and individual variability, and inter-individual variability.

How to effectively featurize the wearable-sensory time series data, while addressing these challenges and achieving the generalization purpose for a multitude of applications can be a tremendous challenge.

A previous body of research in time series has focused on wavelet-based frequency analysis and motif discovery methods. But, these methods each have their own limitations. First, while one can extract discriminating and independent features using wavelet decomposition approaches, it still involves manual effort and domain-specific expert knowledge (e.g., medical knowledge). Second, discovering motifs are computationally expensive and require the repeated process of searching for optimal motifs from candidates. Motivated by these limitations and the success of representation learning for automating feature discovery, recent research has led to representation learning on sensory data. However, this work has focused on representation learning for fixed-length sequential data with high quality complete time series data and face their own set of challenges. First, the wearable-sensory time series data is of variable-length ranging from several days to months, since the actively sensing time period may vary from person to person. Second, readings of sensors are usually missing / incomplete at different time periods for various reasons (e.g., sensor communication errors or power outages). Third, the success of these supervised deep neural network models largely relies on substantial ground truth, which is often scarce.

### BRIEF SUMMARY

Presented herein are various embodiments for analysis of and representation learning for wearable sensor time series data. Such wearable sensor time series data may come, for example, from wearable electronic biometric sensors such as heart rate sensors and monitors.

Embodiments described herein may include systems, methods, and computer program products for analyzing time series data generated by or collected by a wearable sensor. Analyzing time series data generated by or collected by a wearable sensor may include performing an analysis of time series data. Performing an analysis of time series data may include accessing day-specific time series data for a target user, the time series data collected from the target user by a wearable sensor. Performing an analysis of time series data may include encoding the day-specific time series data. Performing an analysis of time series data may include performing a temporal pattern aggregation on the encoded day-specific time series data. Performing an analysis of time series data may include performing Siamese-triplet network optimization on the data aggregation process. Analyzing time series data generated by or collected by a wearable sensor may also include determining an inference from the time series data based upon the analysis

Embodiments presented herein address some or all of the challenges noted above by presenting novel systems, methods, and computer program products for implementing a representation learning algorithm, herein denoted HeartSpace , and brings one closer to the potential of qualified self from quantified self. After describing various embodiments and using two different data data cohorts—captured with different sensors, time periods and locations—it is shown that HeartSpace results in feature representations that are predictive over a number of different tasks, including health states, personality prediction, demographics inference, job performance prediction, and user identification, each achieving higher accuracies than previously existing state-of-the-art systems and methods. As can be appreciated, HeartSpace may also be used to benefit other down-stream applications based on analysis of wearable sensory data, such as disease diagnosis and psychological monitoring. HeartSpace addresses representation learning challenges from such wearable sensor time series data, and demonstrates how physiological response as captured in the heart rate is predictive and indicative of several individual attributes, including demographics and personality.

Presented herein are embodiments that have been developed for a time series representation learning framework, herein denoted HeartSpace, that addresses some or all of the weaknesses and shortcomings of previously known systems and methods for learning effective embeddings given wearable sensor time series data and allows the development of more accurate methods, systems, and computer program products to infer and predict, inter alia, user demographics, personality, work performance attributes, and health states, and other inferences from underlying wearable sensor time series data.

In some embodiments, HeartSpace addresses some or all of the following challenges:
1) Handling variable-length time series and data incompleteness. A straightforward way to address variable-length input is to resize each input time series into a fixed-length vector. However, the resulting vector, derived by interpolation when input length differs from pre-defined length, has less control of variable time series resolutions and fails to capture the consistent time granularity for different individuals, leading to inferior representation. An alternative approach is sequence padding with specific symbols so that all time series are as long as the longest series in the dataset. However, an associated challenge with sequence padding is that all time series data have been artificially created with fixed-length inefficiently. Consequently, artificially created sequences with padding operation may not properly reflect their time-evolving distributions. This is especially critical when trying to draw insights from physiological response data such as heart rate. Thus, a representation learning method should be able to deal with variable length and incomplete data.
2) Differentiating unique patterns from time series data with similar trends. In addition, the wearable-sensory data collected from different people may have very similar distributions. For example, for adults 18 and older, a normal resting heart rate (i.e. number of heart beats per minute) is between 60 and 80 beats per minute. However, there are localized patterns among individuals (intra-sensor and/or individual variability) that offer important nuances on similarities and differences. Thus, a representation learning method should be able to capture the global and local characteristics of such time series data to effectively compute similarities among individuals.
3) Handling data from different kinds of wearable sensors. Different kinds of sensors can have different aspects of recording the physiological data and may have different kinds of measurements (for example, certain wearable devices may compute and record stress measurements or resting heart rate or heart rate variability). It is important for any algorithm to be able to effectively generalize to not only different kind of sensors but also different kind of data and measurements being generated by those sensors.

To summarize, embodiments presented herein provide contributions as follows. One can focus on heart rate as a variable time series data stream as it is rife with the aforementioned challenges and the application space requires accurate and efficient representation learning. Novel and effective systems, methods, and computer program products are developed, herein denoted HeartSpace, that successfully addresses some or all of the aforementioned challenges to creating a generalizable lower-dimensional and latent embedding. In HeartSpace, one may first segment the heart rate data collected from individuals into multiple day-long time series because human behavior in some cases exhibits day-long regularities. In other embodiments, data may be segmented into other arbitrary time windows, including (but not limited to) hourly, weekly, monthly, yearly, among others. Then, a deep autoencoder architecture is developed to map high-dimensional time-specific (i.e., daily) sensor data into the same latent space with a dual-stage gating mechanism. These learned embedding vectors are capable of not only preserving temporal variation patterns, but also largely alleviate the missing data issue of wearable-sensory time series. After that, one may leverage a temporal pattern aggregation network to capture inter-dependencies across time-specific representations based on the developed position-aware multi-head attention mechanism. Both intra-series temporal consistency and inter-series temporal discrepancy are also considered. During the training process, a Siamese-triplet network optimization strategy is designed with the exploration of implicit intra-series temporal consistency and inter-series relations. HeartSpace is evaluated on two real-world wearable-sensory time series datasets representing a diverse group of human subjects and two different sensing devices. Evaluation experiments demonstrate that HeartSpace outperforms previous methods in various applications, including user identification, personality prediction, demographics inference, and job performance prediction, thereby demonstrating the higher quality of the learned embedding produced by embodiments described herein.

The emobdiment described herein provide benefits and advantages and comprehensive solutions toward addressing challenges of analyzing and utilizing time series data collected using wearable sensors by developing novel systems, methods, and computer program products, herein denoted HeartSpace. The utility and effectiveness of the embodiments described herein are demonstrated using at least two real-world data sets representative of different human subjects and environments, and applying to a number of application scenarios.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a training flow of one embodiment of a framework for analysis of wearable sensor time series data.

FIG. 2 illustrates an example architecture of a deep autonencoder module.

FIG. 3 illustrates an example of an architecture of a temporal pattern aggregation network.

FIGS. 4A-4D illustrate examples of length and completeness of time-series data.

FIGS. 5A-5D illustrate examples of data statistics for data with respect to user demographics, personality, and sleep duration.

FIGS. 6A-6B illustrate examples of personality prediction results for two different categories, Agreeableness (6B) and Conscientiousness (6A).

FIGS. 7A-7B illustrate examples of results of job performance prediction.

FIGS. 8A-8B illustrate example performance evaluation results of forecasting sleep duration of people.

FIGS. 9A-9D illustrate results of testing accuracy for various embodiments of analysis methods while eliminating one of a Siamese-triplet network, an autoencoder network, or a multi-head attention mechanism.

FIGS. 10A-10B illustrate a comparison of results from embodiments presented herein compared to those from a previous work, DeepHeart, from a randomly selected 15 participants from a heart rate data set.

FIGS. 11A-11E depicts examples of evaluation results as a function of one selected parameter while keeping other parameters fixed.

FIG. 12 illustrates an exemplary computing system for implementing the systems and methods described herein.

FIG. 13 illustrates an example method for analyzing time series data generated by or collected by a wearable sensor.

### DETAILED DESCRIPTION

Presented herein are various embodiments for analysis of and representation learning for wearable sensor time series data. Such wearable sensor time series data may come, for example, from wearable electronic heart rate sensors and monitors. Such wearable sensors may include, for example (but not be limited to), those produced by Garmin® and Fitbit®. Such wearable sensors may also include any device that can capture streaming data from a body—including chest straps, EKG, pulse-oxymeters, blood glucose monitors, etc.

Embodiments described herein may include systems, methods, and computer program products for analyzing time series data generated by or collected by a wearable sensor. Analyzing time series data generated by or collected by a wearable sensor may include performing an analysis of time series data. Performing an analysis of time series data may include accessing time series data, such as day-specific time series data, for a target user, the time series data collected from the target user by a wearable sensor. Performing an analysis of time series data may include encoding the day-specific time series data. Performing an analysis of time series data may include performing a temporal pattern aggregation on the encoded day-specific time series data. Performing an analysis of time series data may include performing Siamese-triplet network optimization on the aggregated data. Analyzing time series data generated by or collected by a wearable sensor may also include determining an inference and/or a prediction from the time series data based upon the analysis.

### 1 Description of an Exemplary Computing Environment

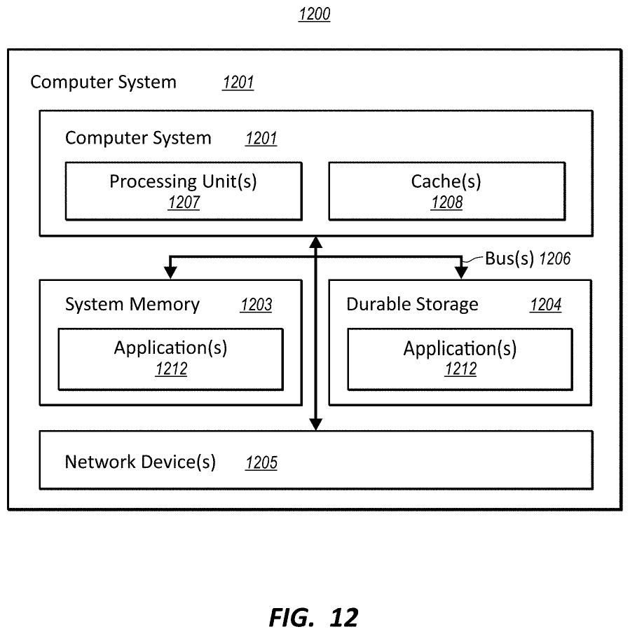

FIG. 12 illustrates an example computing environment 100 that facilitates analysis of and representation learning for wearable sensor time series data. As depicted, computing environment 1200 may comprise or utilize a special-purpose or general-purpose computer system 1201, which includes computer hardware, such as, for example, one or more processors 1202, system memory 1203, durable storage 1204, and/or network device(s) 1205, which are communicatively coupled using one or more communications buses 1206.

Embodiments within the scope of the present invention can include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, embodiments of the invention can comprise at least two distinctly different kinds of computer-readable media: computer storage media and transmission media.

Computer storage media are physical storage media (e.g., system memory 1203 and/or durable storage 1204) that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., network device(s) 1205), and then eventually transferred to computer system RAM (e.g., system memory 1203) and/or to less volatile computer storage media (e.g., durable storage 1204) at the computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, machine code instructions (e.g., binaries), intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud computing model can be composed of various characteristics, such as on-demand self-service, broad network access, resource pooling, rapid elasticity, measured service, and so forth. A cloud computing model may also come in the form of various service models such as, for example, Software as a Service (“SaaS”), Platform as a Service (“PaaS”), and Infrastructure as a Service (“IaaS”). The cloud computing model may also be deployed using different deployment models such as private cloud, community cloud, public cloud, hybrid cloud, and so forth.

Some embodiments, such as a cloud computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

As shown in FIG. 12, each processor 1202 can include (among other things) one or more processing units 1207 (e.g., processor cores) and one or more caches 1208. Each processing unit 1207 may load and execute machine code instructions via the caches 1208. During execution of these machine code instructions at one more execution units, the instructions can use internal processor registers as temporary storage locations and can read and write to various locations in system memory 1203 via the caches 1208. In general, caches 1208 may temporarily cache portions of system memory 1203; for example, caches 1208 might include a “code” portion that caches portions of system memory 1203 storing application code, and a “data” portion that caches portions of system memory 1203 storing application runtime data. If a processing unit 1207 requires data (e.g., code or application runtime data) not already stored in the caches 1208, then the processing unit 1207 can initiate a “cache miss,” causing the needed data to be fetched from system memory 1203—while potentially “evicting” some other data from the caches 1208 back to system memory 1203.

As illustrated, the durable storage 1204 can store computer-executable instructions and/or data structures representing executable software components; correspondingly, during execution of this software at the processor(s) 1202, one or more portions of these computer-executable instructions and/or data structures can be loaded into system memory 1203. For example, the durable storage 1204 is shown as potentially storing computer-executable instructions and/or data structures corresponding to one or more application(s) 1212. The durable storage 1204 can also store data 1210.

The computing system may be communicatively coupled with one or more wearable sensors. The wearable sensors may be, for example, heart rate monitors. The wearable sensors may also sense, generate, and record a vast amount of data, including heart rate, time, date, location, weather, altitude, barometric pressure, GPS data, respiration data, temperature, speed or pace, footsteps, cadence (as in pedal RPM on a bicycle), and various other data. Such wearable sensors include, for example, those produced by Garmin® and Fitbit®.

As may be appreciated, the computing system need not be directly coupled to communicate with a wearable sensor. Data collected by or generated by a wearable sensor may be collected and stored in a location or repository remote from the computing system. For instance, a wearable sensor may have on-board storage for data. A wearable sensor may continuously or periodically download data to another repository. Such repository for data may include Internet or cloud based storage, may include storage on a computing system, and may include any durable data storage available at any location or through any communication medium. In other embodiments, a computing system for performing the analysis described herein may be incorporated within the wearable sensor or included in an integrated system incorporating both a wearable sensor and an analysis system. This may be accomplished, for instance, by a combination of hardware and/or software incorporated within the wearable sensor.

The computing system may access the data collected by or generated by a wearable sensor through various means. The system my access data via local durable storage (e.g., a hard disc, SSD, SIMM card, etc.) or via a network from some remote data storage location (e.g., a local area network, Internet, cloud based storage, etc.).

### 2 Problem Formulation

Preliminary definitions can be introduced and the problem of wearable-sensory time series representation learning can be formalized. Bold capital and bold lower case letters are used to denote matrices and vectors.

Definition 1 Wearable-Sensory Time Series. Suppose there are 1 users (U={u₁, . . . , uᵢ, . . . , u_{I}}), and one may use Xᵢ=(xᵢ¹, . . . , xᵢ^{j}, . . . , xᵢ^{Ji}) (Xᵢ ∈ [symbol] ^{Ji}) to denote the series of temporally ordered wearable-sensory data (e.g., heart rate measurement—the number of times a person's heart beats per minute) of length Jᵢ collected from user uᵢ. In particular, each element xᵢ^{j} represents the j-th measured quantitative value from user uᵢ(1≤j≤Jᵢ and Jᵢ may vary from user to user. Each measurement xᵢ^{j} is associated with a timestamp information tᵢ^{j} and thus one may define a time vector Tᵢ=(tᵢ¹, . . . , tᵢ^{j}, . . . , tᵢ^{Ji}) to record the timestamp information of sequential wearable sensor data Xᵢ.

In reality, since the wearable-sensory data is collected over different time periods (e.g., with different start and end date, different time windows, different durations of data collection, different start and end times within days, etc.), the sequence lengths usually vary among different time series. Additionally, readings of wearable sensors are usually lost at various unexpected moments because of sensor or communication errors (e.g., power outages). Therefore, the wearable-sensory data often exhibits variable-length and missing values.

Problem. Wearable-Sensory Time Series Representation Learning. Given a wearable-sensor time series Xᵢ with variable-length and missing values, the objective is to learn the d-dimensional latent vector Yᵢ ∈ [symbol] ^{d} that is able to capture the unique temporal patterns of time series Xᵢ.

The output of the problem is the low-dimensional vector Yᵢ corresponding to the latent representation of each wearable-sensory time series Xᵢ from user uᵢ. Notice that, although different time series Xᵢ(i ∈ [1, . . . , I]) can be of any length, their representations are mapped into the same latent space. These learned time series representations can benefit various health and wellness tasks without the requirement of substantial training instance pairs.

### 3 Methodology

In one embodiment, HeartSpace may consist of three components:

i) Day-specific time series encoding;

ii) Temporal pattern aggregation; and

iii) Siamese-triplet network optimization.

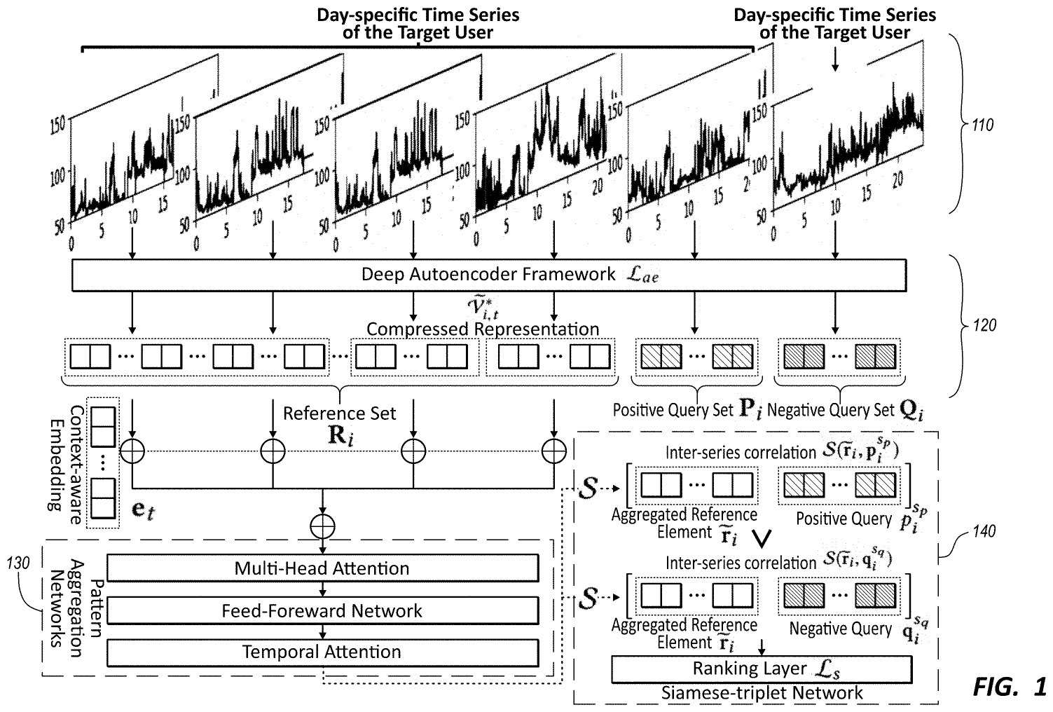

As depicted in FIG. 1, a training flow of one embodiment of a HeartSpace framework may be summarized as:
(1) partition raw sensory data into a set of day-long times series (110);
(2) map each day-long time series into a representation vector by a day-specific time series encoding module (120);
(3) fuse multiple such day specific representations through temporal pattern aggregation network (130); and
(4) update parameters based on reconstruction loss and Siamese-triple loss (140).
   Each of these steps is further elaborated below.

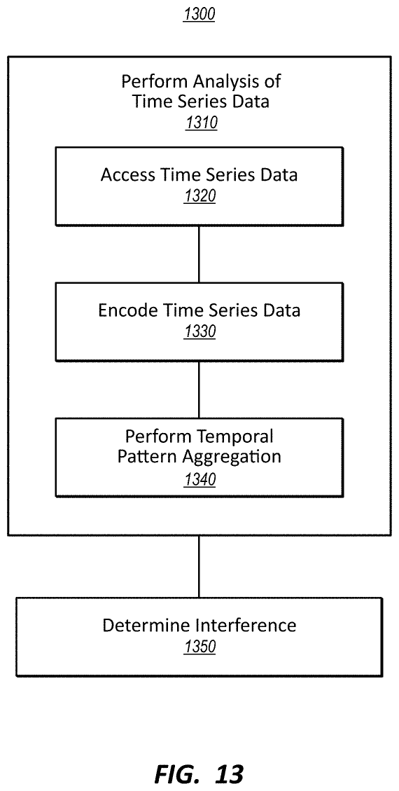

The HeartSpace framework may be used to analyze time series data generated by or collected by a wearable sensor. For example, FIG. 13 depicts a method 1300 for analyzing time series data generated by or collected by a wearable sensor. The method 1300 may include performing an analysis of time series data 1310. Performing an analysis of time series data may include accessing 1320 day-specific time series data for a target user, the time series data collected from the target user by a wearable sensor. Performing an analysis of time series data may include encoding 1330 the day-specific time series data. Performing an analysis of time series data may include performing a temporal pattern aggregation 1340 on the encoded day-specific time series data. Performing an analysis of time series data may include performing Siamese-triplet network optimization 1350 on the aggregated data. Analyzing time series data generated by or collected by a wearable sensor may also include determining an inference 1350 from the time series data based upon the analysis.

Considering that periodicity has been demonstrated as an important factor that governs human sensory data (e.g., heart rate) with time-dependent transition regularities, and the sensed time series data are often variable-length, one may first partition the wearable-sensory time series (i.e., Xᵢ) of each user uᵢ into T (indexed by t) separated day-long time series (T may vary among users).

Definition 2—Day-long Time Series xᵢ^{t}. Each t-th divided day-long time series of Xᵢ is denoted as xᵢ^{t} ∈ [symbol] ^{K}, where K is the number of time steps included in one day. In xᵢ^{t}, each element xᵢ^{t,k} represents the sensed measurement from user uᵢ at the k-th time step in xᵢ^{t}. Due to the data incompleteness issue of the collected time series, one may set the element xᵢ^{t,k} as zero to keep equally-spaced intervals if there is no measurement collected from user uᵢ at the k-th time step in xᵢ^{t}.

### 3.1 Day-Specific Time Series Encoding

To explore the underlying repeated local patterns and reduce dimensions of day-long time series data, one may employ a convolution autoencoder module to map each individual series xᵢ^{t} into a common low-dimensional latent space. In general, the encoder first takes day-long time series as the input and then translates it into a latent representation which encodes the temporal pattern. Then, the decoder network reconstructs the data which is identical with the input xᵢ^{t} in the ideal case. To keep the input dimension consistent with complete day-specific time series, one may apply zero padding by filling in zero value to missing observations. However, it may bring in the negative effect in the encoding process. Since the filled value are treated equally as other valid inputs when apply kernels in each convolutional layer, the generated features may incorrectly encoded and further lead to the error propagation from low to high layers. To mitigate the undesired effects from zero padding, one may use a dual-stage gating mechanism by re-weighting hidden units in each layer. More specifically, each layer of a deep autoencoder framework may be a four-step block:

i) convolution network;

ii) channel-wise gating mechanism;

iii) temporal gating mechanism; and

iv) pooling operation.

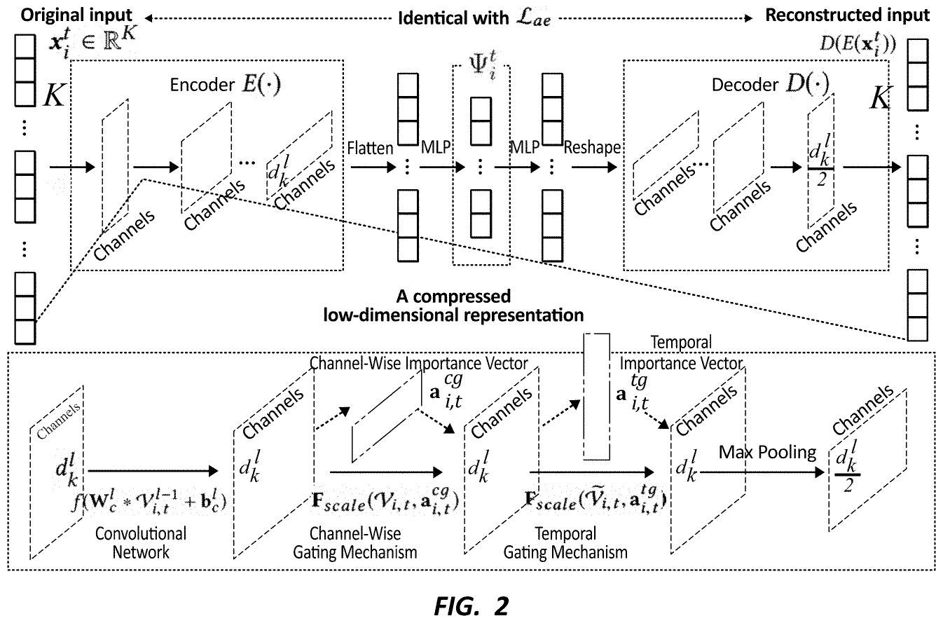

FIG. 2 depicts an example architecture of such a deep autonencoder module.

### 3.1.1 Convolutional Neural Network.

Firstly, one may apply convolutional neural network (CNN) to encode the local pattern of day-long time series xᵢ^{t}. Specifically, feed xᵢ^{t} into a number of convolutional layers. Let's denote v_{i,t}^{l-1} ∈ [symbol] ^{d}^{ₖ}^{^{l-1}}^{×d}^{_{c}}^{^{l-1}} as the feature map representation of (l-1)-th layer, where d_{c}^{l-1}, dₖ^{l-1} indicates the channel dimension, temporal dimension size in (l-1)-th layer, respectively. i and t is user and day index, respectively. Formally, the output of l-th layer in the CNN may be given as:

V_{i,t}^{l}=f(W_{c}^{l}*v_{i,t}^{l-1}+b_{c}^{l}) (1)

where f (·) is the activation function and * denotes the convolutional operation. W_{c}^{l} and b_{c}^{l} represents the transformation matrix and bias term in l-th layer of the CNN, respectively.

### 3.1.2 Channel-Wise Gating Mechanism

In one embodiment of a deep autoencoder module, the goal of the channel-wise gating mechanism is to re-weight hidden units by exploiting the cross-channel dependencies and select the most informative elements from the encoded feature representation V_{i,t}. To exploit the dependencies over channel dimension, one may first apply temporal average pooling operation Fₚₒₒₗ^{cg}(·) on the feature representation V_{i,t} over temporal dimension (1≤k≤K) to produce the summary of each channel-wise representation as:

$${z}_{i , t}^{c g} = {F}_{pool}^{c g} ( {𝒱}_{i , t} ) = \frac{1}{K} {\sum }_{k = 1}^{K} {𝒱}_{[ k . : ]} ; {z}_{i , t}^{cg} ∈ {ℝ}^{{d}_{c}} \qquad ( 2 )$$

where z_{i,t}^{cg} represents the intermediate representation of V_{i,t} after average pooling operation over temporal dimension. Then, a channel-wise gating mechanism recalibrates the information distribution among all elements across channels as:

α_{i,t}^{cg}=Sigmoid(W₂^{cg}·ReLU(W₁^{cg}·z_{i,t}^{cg})); α_{i,t}^{cg} ∈ [symbol] ^{d}^{_{c}} (3)

where a_{i,t}^{cg} denotes the channel-wise importance vector in which each entry is the each channel's importance. W₁^{cg} and W₂^{cg} is the learned transformation matrix of two fully connected neural-net layers. Finally, the channel-wise representations \tilde{V}_{i,t} is learned as:

\tilde{V}_{i,t}=F_{scale}(V_{i,t}, α_{i,t}^{cg})=V_{i,t}⊙α_{i,t}^{cg} (4)

where ⊙ is the element-wise production operation.

### 3.1.3 Temporal-wise Gating Mechanism

To re-weight the hidden units by capturing the temporal dependencies of feature representation across time steps, a gating mechanism is applied on temporal dimension to further learn focus points in the time-ordered internal feature representation \tilde{V}_{i,t} (output from the channel-wise gating mechanism). Similar to the gating mechanism procedures encoded in Equations 2, 3, and 4, the channel average pooling operation is applied on feature representation \tilde{V}_{i,t} over channel dimension and get \tilde{V}*_{i,t} ∈ [symbol] ^{d}^{_{c}} as the summarized feature representation which jointly preserve the channel-wise and temporal dependencies.

### 3.1.4 Encoder-Decoder Configuration

The following presents an architecture configuration of a deep autoencoder module.

Encoder. Given the day-long time series xᵢ^{t}∈ [symbol] ^{K} (for example, K=1440 when a sample interval is one minute (one day=1440 minutes)) , use a Rectifier Linear Unit (ReLU) activation function and contain 5 convolutional layers (i.e., Convl-Conv5) followed by channel-wise, temporal-wise gating mechanism and pooling layers. Particularly, Conv1-Conv5 is configured with the one-dimensional kernel with {9,7,7,5,5} and filter sizes with {32,64,64,128,128}, respectively. Then, perform a flatten operation on the output to generate a one-dimensional feature representation and feed it into a fully connected layer with Tanh activation function to generate the final latent representation Ψᵢ^{t} corresponding to the time series xᵢ^{t}. The number of layers, kernel sizes, and filter sizes are hyperparameters which may be configurable and could vary by specific sample interval (e.g., 30 seconds) of sensory data.

Decoder. The decoder is symmetric to the encoder in terms of layer structure. First, the representation Ψᵢ^{t} is uncompressed by a single-layer with Tanh activation function and then followed by a series of deconvolutional layers with ReLU activation function. The kernel and filter sizes is in reverse order to be symmetric to the encoder architecture configuration. Channel-wise and Temporal-wise are applied in all layers except the last one.

Loss Function in Deep Autoencoder Module: Formally define the reconstruction loss function in the deep autoencoder module as follows:

[symbol]

ₐₑ=∥M⊙(D(E(xᵢ^{t}))−xᵢ^{t}∥₂² (5)

where M ∈ [symbol] is a binary mask vector corresponding to each element in xᵢ^{t}. In particular, Mₖ=1 if xᵢ^{t,k}≠0 (i.e., has measurement) and Mₖ=0 otherwise. ⊙ is the element-wise product operation, xᵢ^{t} is the input, E(·) and D(·) represents the encoder and decoder function.

### 3.2 Temporal Pattern Aggregation Network

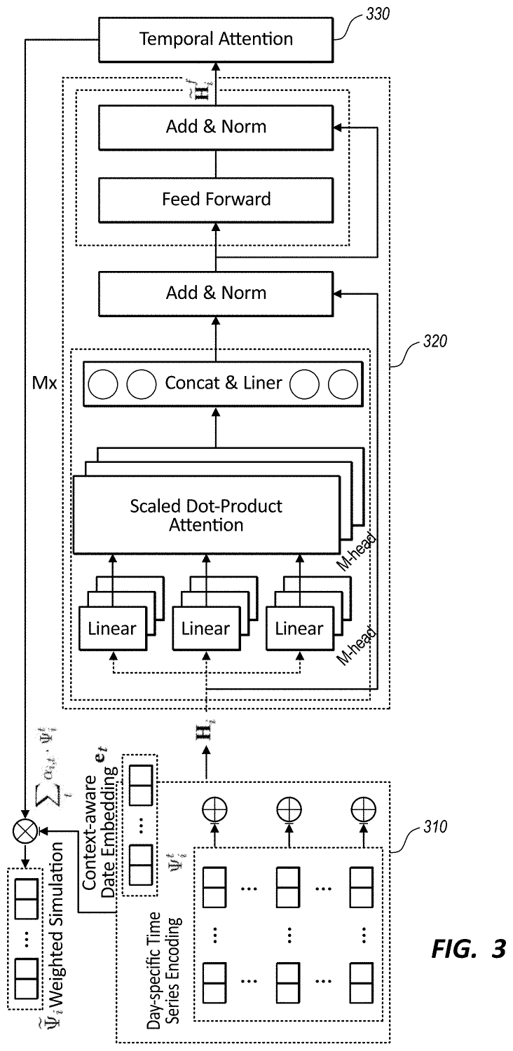

While applying the autoencoder framework to map day-long time series xᵢ^{t} of variable-length into a low-dimensional latent representation Ψᵢ^{t}, how to appropriately fuse day-specific temporal patterns still remains a significant challenge. To address this challenge and aggregate the encoded temporal-wise patterns, a temporal pattern aggregation network is developed which promotes the collaboration of different day-specific temporal units for conclusive cross-time representations. FIG. 3 depicts an architecture of a temporal pattern aggregation network which consists of three major modules:

i) Context-aware time embedding module (310);

ii) multi-head aggregation network (320); and

iii) temporal attention network (330).

### 3.2.1 Context-Aware Time Embedding Module.

From the deep autoencoder module, given the time series Xᵢ of user uᵢ, a set of date-ordered latent representations with size of T, (i.e., Ψᵢ=<Ψ_{i,1}, . . . , Ψ_{i,t}, . . . , ‥_{i,T}>) can be learned. In the real world, different people may exhibit different wearable-sensory data distribution due to distinct or different specific daily routines. To incorporate the temporal contextual signals into the learning framework, the model is augmented with a time-aware embedding module, which utilizes the relative time difference between the last time step and each previous one. For example, given a time series with three date information {Oct. 1, 2018, Oct. 20, 2018, Oct. 25, 2018}, a date duration vector as {24,5,0} (the day duration between Oct. 1, 2018 and Oct. 25, 2018 is 24 days) may be generated. To avoid inconsistency between training and test instances with respect to long date duration, each element of the vector has assigned to it a fix (non-trainable) date embedding using the timing signal method. The embedding vector eₜ of t-th day is derived as:

$${e}_{t , 2 i} = sin ( \frac{t}{{10000}^{2 i / {d}_{e}}} ) ; {e}_{t , 2 i + 1} = cos ( \frac{t}{{100002}^{i / {d}_{e}}} ) \qquad ( 6 )$$

where t is the relative time value and dₑ is the embedding dimension (2i+1 and 2i are the odd and even index in the embedding vector). New context-aware latent vector h_{i,t} ∈ [symbol] ^{d}^{ₑ} is generated by element-wise adding each day-specific feature representation Ψᵢ^{t} and date embedding eₜ, to incorporate the temporal contextual signals into the learned embeddings.

3.2.2 Multi-Head Aggregation Network

During the pattern fusion process, a multi-head attention mechanism is developed which is integrated with a point-wise feedforward neural network layer, to automatically learn the quantitative relevance in different representation subspaces across all context-aware temporal patterns). Specifically, given the i-th time series, all context-aware day-specific embeddings Hᵢ={h_{i,0}, . . . , h_{i,T}} are fed into a multi-head attention mechanism. Here, M-heads attention conducts the cross-time fusion process for M subspaces (m ∈ [1, . . . , M]). Each m-th attention operation involves a separate self-attention learning among Hᵢ as follows:

$${\tilde{H}}_{i}^{m} = softmax ( \frac{{W}_{1}^{m} \cdot {{H}_{i} ( {W}_{2}^{m} \cdot {H}_{i} )}^{T}}{\sqrt{{d}_{m}}} ) {W}_{3}^{m} \cdot {H}_{i} , \qquad ( 7 )$$

where W₁^{m}, W₂^{m}, W₃^{m} ∈ [symbol] ^{d}^{ₘ}^{×d}^{ₑ} represent the learned parameters of m-th head attention mechanism, and dₘ is the embedding dimension of m-th head attention, i.e., dₘ=dₑ/M. Then, concatentate each learned embedding vector \tilde{H}ᵢ^{m} from each m-th head attention, and the cross-head correlations can be captured as follows:

\tilde{H}ᵢ^{c}=W_{c}·concat(\tilde{H}ᵢ¹, . . . , \tilde{H}ᵢ^{m}, . . . , \tilde{H}ᵢ^{m}) (8)

Where W_{c}∈ [symbol] ^{d}^{ₑ}^{×d}^{ₑ} the transformation matrix to model the correlations among head-specific embeddings. Hence, jointly embed multi-modal dependency units into the space with the fused \tilde{H}ᵢ^{c} using the multi-head attention network. The advantage of the multi-head attention network lies in that it allows the exploration of feature modeling in different representation spaces. Then, further feed the fused embedding \tilde{H}ᵢ^{c} into a feed-forward neural network:

\tilde{H}ᵢ^{f}=W₂^{f}·ReLU(W₁^{f}·\tilde{H}ᵢ^{c}+b₁^{f})+b₂^{f}, (9)

where W₁^{f}, W₂^{f} and b₁^{f}, b₂^{f} are the weight matrix and bias in the feed-forward layer. In a particular embodiment of the HeartSpace framework, the multi-head attention mechanism is performed twice, which can be varied in terms of different sensory data distributions.

3.2.3 Temporal Attention Network

To further summarize the temporal relevance, a temporal attention network is developed to learn importance weights across time. Formally, the temporal attention module can be represented as follows:

αᵢ=softmax(c·Tanh(W^{α}·\tilde{H}ᵢ^{f}+b^{α}));\tilde{Ψ}ᵢ=Σₜα_{i,t}·Ψᵢ^{t} (10)

Output \tilde{H}ᵢ^{f} is first fed into a one-layer neural network and then together with the context vector c to generate the importance weights αᵢ through the softmax function. The aggregated embedding \tilde{Ψ}ᵢ is calculated as a weighted sum of day-specific embeddings based on the learned importance weights. For simplicity, denote the temporal pattern aggregation network may be denoted as \tilde{Ψ}ᵢ=A(Ψᵢ¹, . . . , Ψᵢ^{T}).

### 3.3 Siamese-triplet Network Optimization Strategy

A goal can be to embed each individual wearable-sensory time series into low-dimensional spaces, in which every time series is represented as an embedding vector. A Siamese-triplet network optimization strategy is developed to jointly model the structural information of intra-series temporal consistency and inter-series correlations in the learning process. In particular, within a series of sensing data points (e.g., heart rate records), even though the measurements change over time, they do not change drastically across multiple days, since they belong to the same user. Additionally, the heart rate measurements sampled from consecutive time intervals (e.g., days) of the same people may a more similar distribution as compared to sampling from different people. Motivated by these observations, a key idea of a Siamese-triplet network optimization framework is to learn representations with the constrain—making intra-series data point pairs closer to each other and inter-series data point pairs further apart. The following terms may be defined to be used in an optimization strategy.

Definition 3—Reference Set Rᵢ: define Rᵢ to represent the sampled reference set of user uᵢ. In particular, Rᵢ={rᵢ¹, . . . , rᵢ^{S}^{ᵣ}}, where Sᵣ is size of reference set corresponding to Sᵣ sampled day-specific time series from Xᵢ. Each entry in Rᵢ represents the sᵣ-th sampled time series.

Definition 4—Positive Query Set Pᵢ: define Pᵢ={pᵢ¹, . . . , pᵢ^{S}^{ₚ}} to denote the positive query set of user with size of Sₚ. Specifically, every entry pᵢ^{s}^{ₚ} represents the sₚ-th sampled day-specific time series xᵢ^{t} from user uᵢ.

Definition 5—Negative Query Set Qᵢ: Qᵢ is defined to as the negative query set for user uᵢ. Particularly, Qᵢ={qᵢ¹, . . . , qᵢ^{S}^{_{q}}} where qᵢ^{s}^{_{q}} is the sampled s_{q}-th day-specific time series from other users except user u_{i′}, i.e., u_{i′}(i′≠i).

Based on the above definitions, given a specific user uᵢ, estimate the similarity may be estimated between his/her reference set rᵢ and each query from his/her positive query set Pᵢ and negative query set Qᵢ. In particular, first aggregate the elements from user uᵢ's reference set Sᵢ as: \tilde{r}ᵢ=A(Rᵢ), where A(·) is the aggregation function which represents the developed temporal pattern aggregation network. Then, compute the cosine similarity of aggregated reference element \tilde{r}ᵢ and each query (i.e., pᵢ^{s}^{ₚ} and qᵢ^{s}^{_{q}}) from the generated positive query set Pᵢ and negative query set Qᵢ. Formally, the similarity estimation function [symbol] is presented as follows:

S(\tilde{r}ᵢ, pᵢ^{s}^{ₚ})=\tilde{r}ᵢ·(E(pᵢ^{s}^{ₚ}))^{T}; sₚ ∈ [1, . . . , Sₚ];

S(\tilde{r}ᵢ, qᵢ^{s}^{_{q}})=\tilde{r}ᵢ·(E(qᵢ^{s}^{_{q}}))^{T}; s_{q} ∈ [1, . . . , S_{q}]; (11)

where S(\tilde{r}ᵢ, pᵢ^{s}^{ₚ}) ∈ [symbol] and S (\tilde{r}ᵢ, qᵢ^{s}^{_{q}}) ∈ [symbol] . To capture the temporal consistency of each individual user uᵢ and inconsistency among different users, a goal is to learn latent representations which preserve inherent relationships between each user's reference set and query set, i.e., time series embeddings from the same user are closer to each other, while embeddings from different users are more differentiated from each other. More specifically, the similarity between the aggregated reference element \tilde{r}ᵢ and the positive query pᵢ^{s}^{ₚ} should be larger, while the similarity between \tilde{r}ᵢ and the negative query qᵢ^{s}^{_{q}} should be smaller, i.e., S(\tilde{r}ᵢ, pᵢ^{s}^{ₚ})>S( [symbol] ᵢ, qᵢ^{s}^{_{q}}). Therefore, a loss function may be formally defined as follows:

[symbol]

ₛ=max(0, S(\tilde{r}ᵢ, qᵢ^{s}^{_{q}})−S(\tilde{r}ᵢ, pᵢ^{s}^{ₚ})+z) (12)

where z is the margin between two similarities. The objective function of joint model is defined as: [symbol] ⱼₒᵢₙₜ= [symbol] ₐₑ+λ [symbol] ₛ, where λ is the coefficient which control the weight of Siamese-triplet loss. The model parameters can be derived by minimizing the loss function. An Adam optimizer may be used to learn the parameters of HeartSpace.
The model optimization process may be summarized as in Algorithm 1.

Algorithm 1: The Model Inference Process of HeartSpace.

| | Input: User set U, batch size b_{size}, support set size Sᵣ, positive query size Sₚ, negative query |
|---|---|
| | size S_{q} |
| 1 | Initialize all parameters; |
| 2 | foreach batch do |
| 3 | sample a set of b_{size} users V from User set U; |
| 4 | foreach user uᵢ in V do |
| 5 | sample support set Rᵢ and positive set Pᵢ from Xᵢ; |
| 6 | sample negative set Qᵢ from Xⱼ ; |
| 7 | feed each entry in Rᵢ, Pᵢ and Qᵢ into autoencoder to get daily representations and |
| | compute [symbol] ₐₑ; |
| 8 | aggregate support set Rᵢ and derive Lₛ; |
| 9 | end |
| 10 | update all parameters w.r.t [symbol] ⱼₒᵢₙₜ = [symbol] ₐₑ + λ [symbol] ₛ; |
| 11 | end |

Unsupervised and semi-supervised learning scenarios. HeartSpace is a general representation learning framework that is flexible for both unsupervised (without labeled training instances) and semi-supervised (limited number of labeled instances) learning. In semi-supervised learning, given the labeled time series and its target value, the learned representation vector may be taken as the input of a single-layer perceptrons with a combined loss function, i.e., integrate the joint objective function [symbol] ⱼₒᵢₙₜ with the loss function based on cross-entropy (categorical values) or MSE (quantitative values).

### 4 Demonstration and Evaluation of Benefits and Advantages

To measure performance as compared to prior systems and methods, various embodiments of HeartSpace were comprehensively evaluated on several inference and prediction tasks: user identification, personality prediction, demographic inference, and job performance prediction. Longitudinal real-world data was used from two different studies leveraging two different sensors, Garmin and Fitbit, and different population groups, thus allowing performance to be carefully vetted and findings validated. To robustly evaluate the accuracy and generalization of embeddings learned by HeartSpace, within the context of the aforementioned prediction tasks, the following questions were considered.
Q1: How does HeartSpace perform compared with previous state-of-the-art representation learning methods for the wearable-sensory time series, as represented by heart rate?
Q2: How is the performance of HeartSpace with respect to different training/testing time periods in user identification?
Q3: How do each of the different components of HeartSpace (i.e., deep autoencoder module, multi-head aggregation network and Siamese-triplet network optimization strategy) contribute to the HeartSpace performance?
Q4: How do key hyperparameters (e.g., reference set size Sᵣ and embedding dimension dₑ) affect HeartSpace performance?
Q5: How does HeartSpace lend itself for visualizing similarities and differences among individuals (interpretation)?

### 4.1 Experimental Settings

### 4.1.1 Data Description

To determine the effectiveness of HeartSpace, heart rate time series data from two separate research projects was evaluated. The time series data is denoted herein based on the sensor used to collect the experimental data: Garmin and Fitbit, respectively.

Garmin Heart Rate Data. This dataset came from an on-going research study of workplace performance which measures the physiological states of employees in multiple companies. This dataset is collected from 578 participants (age between 21 to 68) by a Garmin sensor band from March 2017 to August 2018. Each measurement is formatted as (user id, heart rate, timestamp).

Fitbit Heart Rate Data. This dataset is collected from a research project at a university which aims to collect survey and wearable data from an initial cohort of 698 students (age between 17 to 20) who enrolled in the Fall semester of 2015. This dataset was collected by Fitbit Charge sensors during the 2015-2016 and 2016-2017 academic years.

### 4.1.2 Data Distribution

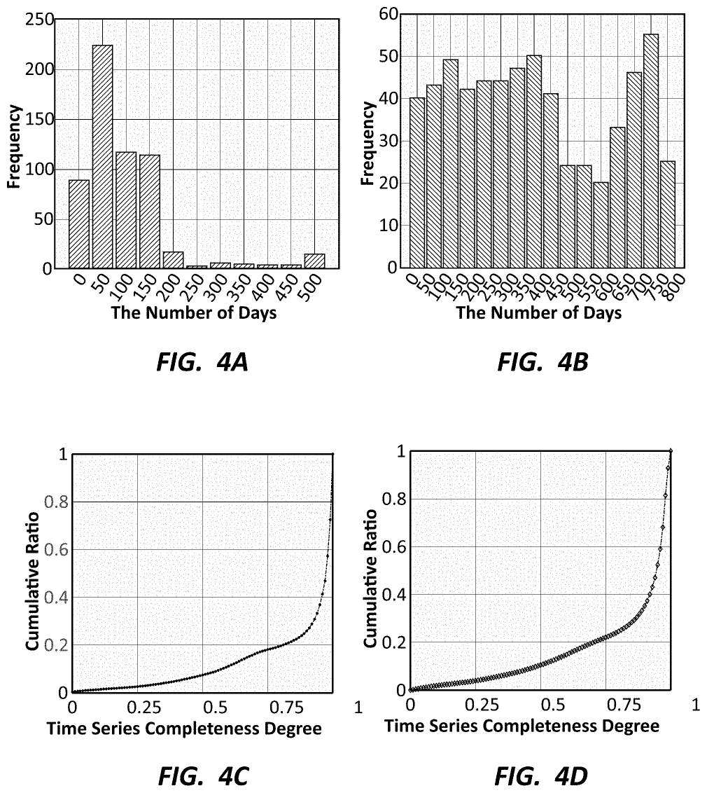

The distribution of wearable-sensory time series is shown in FIGS. 4A-4D in terms of time series length Jᵢ and completeness degree (i.e., the ratio of non-zero elements in day-specific time series xᵢ^{t}) on both Garmin and Fitbit heart rate data. As depicted in FIGS. 4A and 4B, different datasets have different time series distributions. Furthermore, FIGS. 4C and 4D illustrate that data incompleteness is ubiquitous, e.g., there exists more than 20% day-specific time series with data incompleteness<0.8, which poses a further challenge for HeartSpace. These observations are a motivation to develop a temporal pattern aggregation network and a dual-stage gating mechanism for handling variable-length time series with incomplete data.

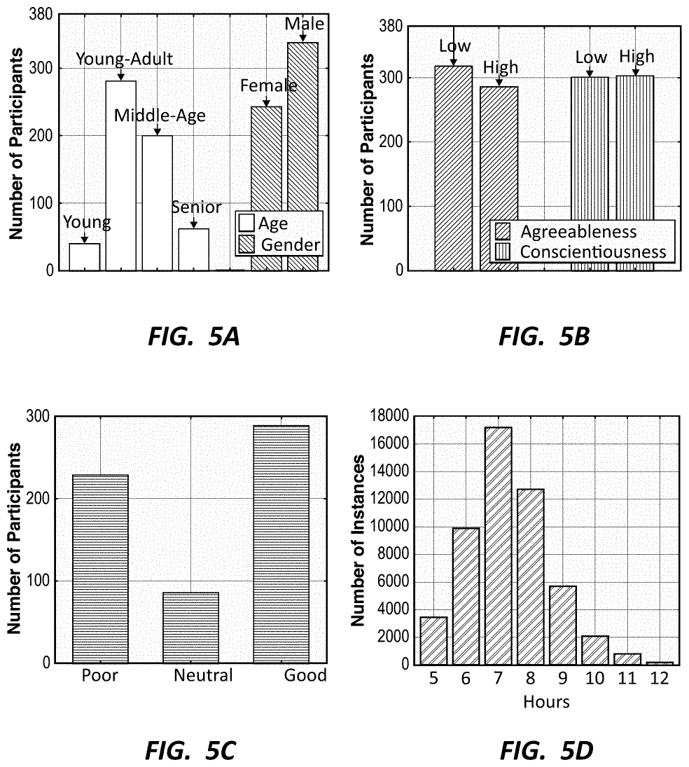

FIGS. 5A-5D illustrate examples of data statistics for data with respect to user demographics, personality, and sleep duration. FIG. 5A illustrates distributions of participants for age and for gender. FIG. 5B illustrates distributions of participants for two personality traits, agreeableness and conscientiousness. FIG. 5C illustrates distributions of participants for job performance rated within three categories, poor, neutral, and good. FIG. 5D illustrates distributions of participants for sleep duration, broken down into hourly groups from 5 hours to 12 hours.

### 4.1.3 Methods for Comparison

To justify the effectiveness of HeartSpace for representation learning on wearable-sensory data, results were compared with the following contemporary representation learning methods:
Convolutional Autoencoder (CAE): CAE is a representation learning framework by applying convolutional autoencoder to map the time series patterns into latent embeddings.
Deep Sequence Representation (DSR): DSR is a general-purpose encoder-decoder feature representation model on sequential data with two deep LSTMs, one to map input sequence to vector space and another to map vector to the output sequence.
Multi-Level Recurrent Neural Networks (MLR): MLR is a multi-level feature learning model to extract low- and mid-level features from raw time series data. RNNs with bidirectional-LSTM architectures are employed to learn temporal patterns.
Sequence Transformer Networks (STN): STN is an end-to-end trainable method for learning structural information of clinical time-series data, to capture temporal and magnitude invariances.
Wave2Vec: Wave2Vec is a sequence representation learning framework using skip-gram model by considering sequential contextual signals. Specifically, it takes the one-by-one data points that surround the target point within a defined window, to feed into a neural network for appear probability prediction. The window size and number of negative samples are set as 1 and 4, respectively.
DeepHeart: DeepHeart is a deep learning approach which models the temporal pattern of heart rate time-series data with the integration of the convolutional and recurrent neural network architecture.
   (The initial parameter settings for each of the above methods are consistent with their original papers.)

### 4.1.4 Evaluation Protocols

To measure the effectiveness of HeartSpace for representation learning on heart rate data in various multi-class classification and binary-class classification tasks, two sets of popular evaluation metrics were adopted in the evaluation. In particular, the first set of metrics are used to evaluate the accuracy of binary-class classification tasks in terms of FI-score, Accuracy and AUC. The second set of metrics are used to evaluate the classification performance on multi-class in terms of Macro-F1and Micro-F1. Details for four different tasks are summarized as follows:
User Identification: In the user identification evaluation, each of the aforementioned methods learns a mapping function to encode each of the day-specific time series data into a low-dimensional representation vector. Then, given multiple day-specific time series data from one user and an unknown day-specific time series, the task is to predict whether the unknown time series is collected from the same user of the multiple day-specific time series. Specifically, first map day-specific time series to embedding vector by utilizing each of the aforementioned methods. Then aggregate embedding vectors of multiple day-specific time series to generate the reference vector. Apply mean-pooling operation on baselines and temporal aggregation network on HeartSpace during the aggregation process. After that, take the element-wise product between the reference vector and the embedding vector of the single day-specific time series as the input. Here, adopt a logistic regression classifier to learn the model from the training data and generate the prediction. The True Positives and True Negatives are the heart rate series that are correctly identified for user uᵢ or not, respectively, by the classifier. The False Positives and False Negatives are the heart rate series that are misclassified as belonging to user uᵢ or not, respectively.
Personality Prediction: Two personality attributes of the participants were considered, namely Conscientiousness and Agreeableness. A classification task of a binary prediction on these attributes was considered—high or low for each participant uᵢ. In this task, the goal was to evaluate whether the embeddings learned from the heart rate are effective predictors of the personality types. A Logistic Regression classifier was used to make the prediction with the embedding as the feature space. The True Positives and True Negatives are the participants that are correctly classified by the classification method as high- and low-level, respectively. The False Positives and False Negatives are the high- and low-level participants that are misclassified, respectively. Only the Garmin data was used for this task (as personality attributes were available only for the subjects in the pool).
User Demographic Inference: It was evaluated whether user demographics, such as gender and age are predictable by using a similar experimental construct. The age information is categorized into four categories (i.e., Young: from 18 to 24, Young-Adult: from 23 to 34, Middle-age: from 39 to 49, and Senior: from 49 to 100).
Job Performance Prediction: The accuracy of predicting a participant's job performance (Individual Task Performance), which is categorized into three types, i.e., good, neutral, and poor, were evaluated. Only the Garmin data was used for this task, as job performance information was available only for the subjects in the Garmin pool.
   User identification and personality prediction tasks are evaluated in terms of F1-score, Accuracy and AUC. In addition, the user demographic inference and job performance prediction tasks, given multi-class scenario, are evaluated using Macro-F1 and Micro-F1.

### 4.1.5 Training/Test Data Split

The details of training/test data partition for different evaluation tasks are summarized as follows:
For demographic inference, personality prediction and job performance prediction task, the entire heart rate data was used for learning time series embeddings, and the labels were split with 60%, 10% and 30% for training, validation and test, respectively.
For the user identification task, one may conduct the HeartSpace's model inference and user identification process are conducted separately and the datasets are split in chronological order. First use Garmin data from March to December in 2017 (10 months), to learn parameters of HeartSpace for time series embedding in an unsupervised fashion. Then, leverage the data from January 2018 to August 2018 to evaluate the user identification performance based on the generated embeddings from the learned model. Specifically, perform the training/test process is performed over the period of January 2018 to August 2018 with a sliding window of two months, i.e., (January→training, February→test); . . . ; (July→training, August→test). The training month provides the labels to learn the classifier and the test month is used to evaluate the prediction accuracy. Irrespective of the training & test month combination, the model parameters are learned on the basis of 2017 data in an unsupervised fashion. This allows the generalization of performance over time to be considered. Moreover, to ensure the fairness of performance comparison, the day-long time series of users shown in the test set are not visible in the training set using the partition strategy. The training/test partition method on Fitbit data is similar to the Garmin data.

### 4.2 Reproducibility

All the deep learning baselines and the proposed HeartSpace framework were implemented using a Tensorflow source platform for machine learning. For the sake of fair comparison, all experiments were conducted across all participants in the testing data and the average performance is reported. Furthermore, the validation was run ten times and the average performance numbers are reported. (The code is available from the inventors.)

For the sake of reproducibility, the hyperparameter settings of HeartSpace are summarized in Table 1. In the experiments, a Glorot initialization was utilized and grid search strategies for hyperparameter initialization and tuning of all compared methods was utilized. Early stopping is adopted to terminate the training process based on the validation performance. After parameter tuning on all baselines, their best performance in the evaluation results is reported. During the model learning process, an Adam optimizer was used for all gradient-based methods, where the batch size and learning rate were set as 64 and 0.001, respectively.

TABLE 1
Parameter Settings

| Parameter | Valuee | Parameter | Valuee |
|---|---|---|---|
| Embedding Dimension | 64 | Support Size | 6 |
| Positive Sampling Size | 2 | Negative Sampling Size | 4 |
| Margin Value | 1 | Siamese-triplet weight | 0.1 |
| Batch Size | 64 | Learning Rate | 0.001 |

### 4.3 Performance Comparison (Q1 and Q2)

### 4.3.1 User Identification

Table 2 shows the user identification performance on both datasets. One can observe that HeartSpace achieves the best performance and obtains significant improvement over state-of-the-art methods in all cases. This sheds lights on the benefit of HeartSpace , which effectively captures the unique signatures of individuals. Although other neural network-based methods preserve the temporal structural information to learn latent representations for each individual time series, they ignore the variable-length and incompleteness of wearable-sensory data, which reveals the practical difficulties in learning accurate models across non-continuous time steps.

TABLE 2
User identification performance in terms of F1-score, Accuracy and AUC.

| Month | Feb | | | Apr | | | June | | | Aug | | |
|---|---|---|---|---|---|---|---|---|---|---|---|---|
| Method | F1-score | Accuracy | AUC | F1-score | Accuracy | AUC | F1-score | Accuracy | AUC | F1-score | Accuracy | AUC |
| Data | Garmin Heart Rate Data | | | | | | | | | | | |
| CAE | 0.6584 | 0.6505 | 0.7112 | 0.6809 | 0.6728 | 0.7378 | 0.6602 | 0.6696 | 0.7425 | 0.6841 | 0.6980 | 0.7705 |
| DSR | 0.6496 | 0.6433 | 0.6926 | 0.6851 | 0.6700 | 0.7270 | 0.7086 | 0.7080 | 0.7841 | 0.6948 | 0.6964 | 0.7722 |
| MLR | 0.6524 | 0.6379 | 0.6859 | 0.6898 | 0.6708 | 0.7316 | 0.7075 | 0.7028 | 0.7739 | 0.7032 | 0.7046 | 0.7765 |
| STN | 0.6161 | 0.5727 | 05984 | 0.7322 | 0.7372 | 0.8169 | 0.7504 | 0.7585 | 0.8434 | 0.7330 | 0.7406 | 0.8176 |
| Wave2Vec | 0.7431 | 0.7515 | 0.8327 | 0.7969 | 0.8034 | 0.8844 | 0.8164 | 0.8223 | 0.9014 | 0.8246 | 0.8301 | 0.9090 |
| DeepHeart | 0.7313 | 0.7384 | 0.8125 | 0.7537 | 0.7579 | 0.8349 | 0.7663 | 0.7730 | 0.8558 | 0.7507 | 0.7567 | 0.8446 |
| HeartSpace | 0.7646 | 0.7649 | 0.8410 | 0.8050 | 0.8068 | 0.8904 | 0.8303 | 0.8335 | 0.9135 | 0.8587 | 0.8621 | 0.9352 |
| Data | Fitbit Heart Rate Data | | | | | | | | | | | |
| CAE | 05794 | 0.5767 | 0.6023 | 0.5961 | 0.5942 | 0.6405 | 0.6023 | 05869 | 0.6335 | 0.6060 | 0.5996 | 0.6387 |
| DSR | 0.6478 | 0.6467 | 0.6962 | 0.6304 | 0.6177 | 0.6547 | 0.6191 | 0.6129 | 0.6719 | 0.6136 | 0.5984 | 0.6411 |
| MLR | 0.6431 | 0.6395 | 0.6977 | 0.6266 | 0.6244 | 0.6808 | 0.6737 | 0.6654 | 0.7400 | 0.6596 | 0.6497 | 0.7051 |
| STN | 0.7123 | 0.7186 | 0.7789 | 0.6877 | 0.6896 | 0.7496 | 0.6868 | 0.6811 | 0.7462 | 0.7297 | 0.7276 | 0.7875 |
| Wave2Vec | 0.7785 | 0.7783 | 0.8626 | 0.7775 | 0.7771 | 0.8573 | 0.7713 | 0.7705 | 0.8572 | 0.8136 | 0.8121 | 0.8938 |
| DeepHeart | 0.6784 | 0.6805 | 0.7462 | 0.7155 | 0.7186 | 0.7878 | 0.7196 | 0.7228 | 0.7916 | 0.7155 | 0.7143 | 0.7842 |
| HeartSpace | 0.7997 | 0.7977 | 0.8696 | 0.8041 | 0.8037 | 0.7869 | 0.7869 | 0.7826 | 0.8680 | 0.8330 | 0.8315 | 0.9147 |

### 4.3.2 Demographic Inference

The demographic inference performance comparison between HeartSpace and other competitive methods on the Garmin heart rate data is shown in Table 3. It can be noted that HeartSpace outperforms other baselines in inferring users' age and gender information, which further demonstrate the efficacy of HeartSpace in learning significantly better time series embeddings than existing state-of-the-art methods. Similar results can be observed for the Fitbit heart rate data. In summary, an advantage of HeartSpace lies in its proper consideration of comprehensive temporal pattern fusion for time series data.

TABLE 3
User demographic inference results.

| Demographic | Age | | Gender | |
|---|---|---|---|---|
| Metric | Micro-F1 | Macro-F1 | Micro-F1 | Macro-F1 |
| CAE | 0.5174 | 0.3644 | 0.6744 | 0.6558 |
| DSR | 0.5058 | 0.2590 | 0.6453 | 0.6334 |
| MLR | 0.5233 | 0.2704 | 0.6453 | 0.6281 |
| STN | 0.5291 | 0.3460 | 0.6279 | 0.6228 |
| Wave2Vec | 0.5465 | 0.3540 | 0.6570 | 0.6455 |
| DeepHeart | 0.4942 | 0.2664 | 0.6395 | 0.6189 |
| HeartSpace | 0.5581 | 0.3773 | 0.7035 | 0.6724 |

### 4.3.3 Personality Prediction

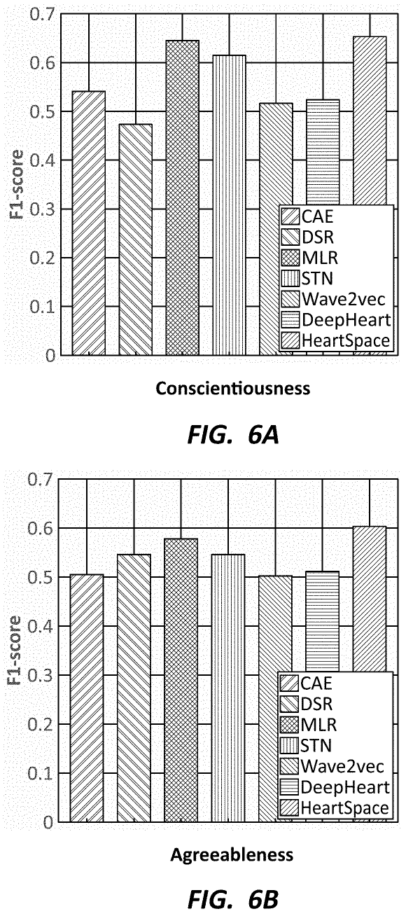

FIGS. 6A and 6B show personality prediction results on two different categories (i.e., Agreeableness (6B) and Conscientiousness (6A)) using each of CAE, DSR, MLR, STN, Wave3Vec, DeepHeart, and HeartSpace, and comparing the results of each. FIGS. 6A and 6B illustrate that HeartSpace, as described herein, achieves the best performance in all personality cases. The performance is followed by DSR which extracts both multi-level temporal features during the representation learning process. This further verifies the utility of temporal pattern fusion in mapping time series data into common latent space.

### 4.3.4 Job Performance Prediction

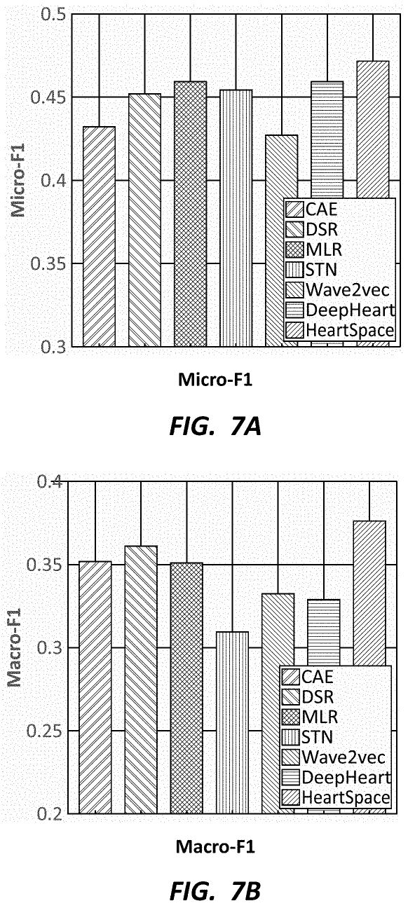

The results of job performance prediction using each of CAE, DSR, MLR, STN, Wave3Vec, DeepHeart, and HeartSpace, are presented in FIGS. 7A and 7B. In these figures, one can notice that HeartSpace achieves the best performance in terms of both Macro-F1 (7B) and Micro-F1 (7A).

### 4.3.5 Sleep Duration Inference

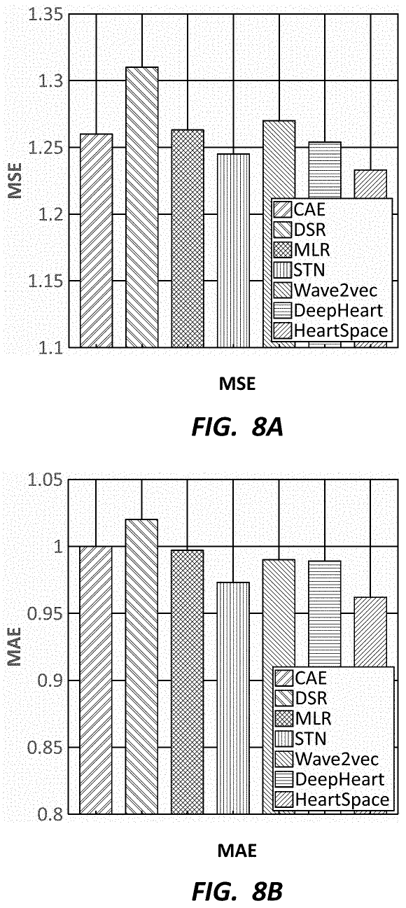

HeartSpace was further evaluted with the application of forecasting the sleep duration of people. The performance evaluation results (measured by MSE (8A) and MAE (8B)) using each of CAE, DSR, MLR, STN, Wave3Vec, DeepHeart, and HeartSpace, are presented in FIGS. 8A (MSE) and 8B (MAE). One can observe that significant performance improvements are consistently obtained by HeartSpace over other previously state-of-the-art baselines, which further validates the effectiveness and benefits of HeartSpace.

### 4.4 Model Ablation: Component-Wise Evaluation of HeartSpace (Q3)

One may also get a better understanding of various components of HeartSpace. In particular evaluations, three variants of the HeartSpace systems and methods described herein were considered, each corresponding to different analytical aspects:
Effect of Siamese-triplet Network HeartSpace-s: A simplified version of HeartSpace which does not include Siamese-triplet network to model intra- and inter-time series inter-dependencies.
Effect of Deep Autoencoder Module. HeartSpace-a: A simplified version of HeartSpace without deep autoencoder module, i.e., only consider Lₛ (Siamese-triplet Network) in the loss function.
Effect of Multi-head Attention Network. HeartSpace-h: A variant of HeartSpace without the multi-head attention network to learn the weights of different day-specific temporal patterns.

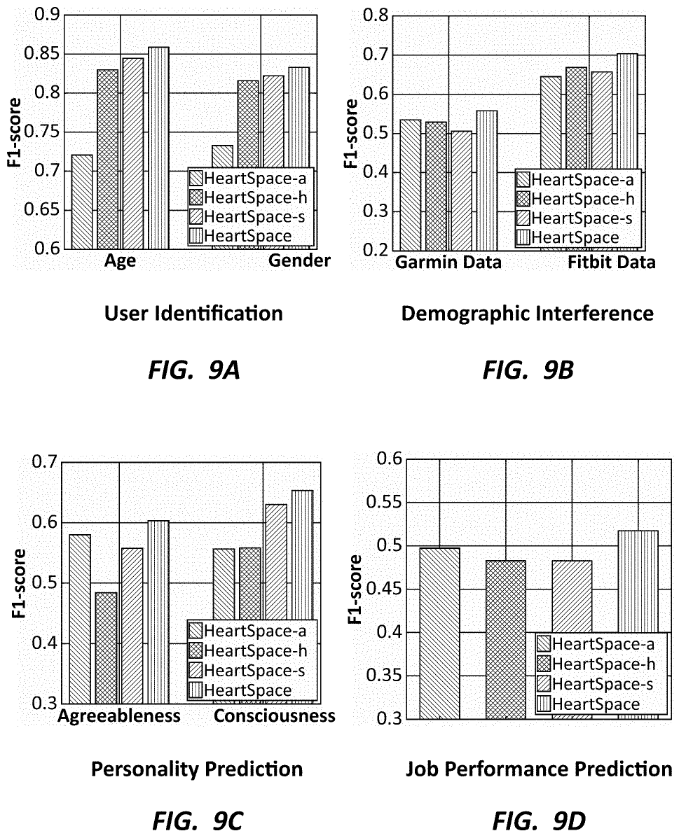

The results of the above evaluations are depicted in FIGS. 9A, 9B, 9C, and 9D. The illustrated F1-score is a measure of the accuracy when using each of the full version of HeartSpace and the simplified versions HeartSpace-a, HeartSpace-h, and HeartSpace-s for each of user identification for age and gender (9A), demographic interference (9B), prediction of the personality traits agreeableness and consciousness (9C), and prediction of job performance (9D). One may notice that the full version of HeartSpace, including each of the multi-head attention network, the deep autoencoder module, and the Siamese-triplet network, achieves the best performance in all cases over each of the simplified versions HeartSpace-a, HeartSpace-h, and HeartSpace-s. These results imply:
(i) The increased efficacy of the designed Siamese-triplet network optimization strategy for preserving structural information of implicit intra- and inter-time series correlations.
(ii) The increased effectiveness of HeartSpace in capturing complex temporal dependencies across time steps for variable-length sensor data.
(iii) The increased effectiveness of HeartSpace in exploring feature modeling in different representation spaces during the pattern fusion process. As such, it is necessary to build a joint framework to capture multi-dimensional correlations in wearable-sensory time series representation learning.

### 4.5 Hyperparameter Studies (Q4)

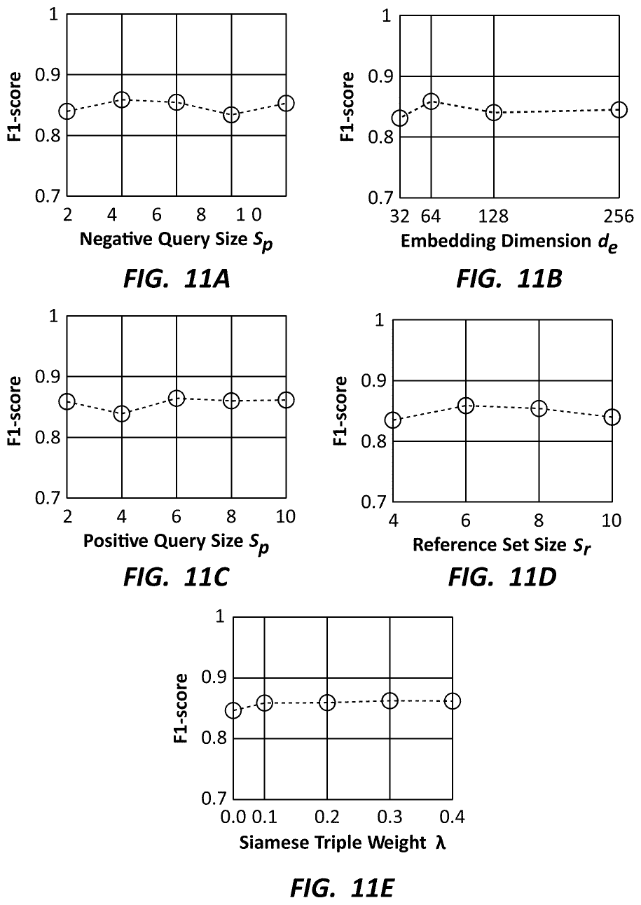

To demonstrate the robustness of the HeartSpace framework, one can examine how the different choices of five key parameters affect the performance of HeartSpace. Except for the parameter being tested, other parameters were set at the default values (see Table 1). FIGS. 11A-11E show evaluation results as a function of one selected parameter while keeping the other parameters fixed. Overall, it can be observed that HeartSpace is not strictly sensitive to these parameters and is able to reach high performance under a cost-effective parameter choice. This demonstrates the robustness of HeartSpace.

Furthermore, One can observe that the increase of prediction performance saturates as the representation dimensionality increases. This is because: at the beginning, a larger value of embedding dimension brings a stronger representation power for the recent framework, but the further increase of dimension size of latent representations might lead to the overfitting issue. In experiments, one can set the dimension size as 64 due to the consideration of the performance and computational cost. One can observe that both the positive query set size and negative query set size, as well as siamese triple loss coefficient have a relatively low impact on the model performance.

### 4.6 Case Study: Visualization (Q5)

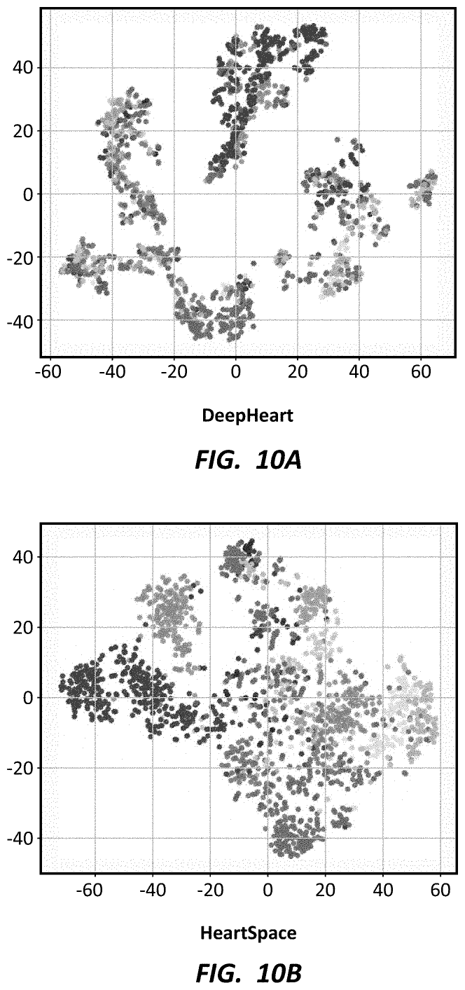

A TensorFlow embedding projector was employed to further visualize the low-dimensional time series representations learned by HeartSpace and one selected baseline (DeepHeart) on Garmin heart rate dataset. FIGS. 10A and 10B show a visualization of HeartSpace (10B) as compared to DeepHeart 2D t-SNE projections (10A) of 64D embeddings of all day-specific time series embeddings from a randomly selected 15 participants from the Garmin heart rate data. It can be observed that the embeddings from the same user can be identified by HeartSpace (10B) and cluster them closer than other embeddings, while the embeddings learned by DeepHeart (10A) of different users are mixed and cannot be well identified. Therefore, the HeartSpace model generates more accurate feature representations of user's time series data and is capable of preserving the unique signatures of each individual, which can then be leveraged for other similarity analyses.

These experiments and evaluations, inter alia, clearly demonstrate the effectiveness and improvement of the embodiments of HeartSpace as described herein over previously known methods and systems.

### Conclusion

Herein has been presented various embodiments for a time series representation learning framework for analyzing wearable-sensory data that addresses several challenges stemming from such data and overcomes particular limitations of previously state-of-the-art approaches, including dealing with incomplete and variable length time series data, intra-sensor/individual variability, and absence of ground truth. Embodiments herein first learn latent representation to encode temporal patterns of individual day-specific time series with a deep autoencoder model. Then, an integrative framework of a pattern aggregation network and a Siamese-triplet network optimization maps variable-length wearable-sensory time series into the common latent space such that the implicit intra-series and inter-series correlations well preserved. Extensive experiments have demonstrated that the latent feature representations learned by embodiments presented herein are significantly more accurate and generalizable than previous methods.

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. When introducing elements in the appended claims, the articles “a,” “an,” “the,” and “said” are intended to mean there are one or more of the elements. The terms “comprising,” “including,” and “having” are intended to be inclusive and mean that there may be additional elements other than the listed elements.

What is claimed:

1. A method, implemented at a computer system that includes at least one processor, for analyzing time series data from a wearable sensor, the method comprising:
performing an analysis of time series data by:
accessing day-specific time series data for a target user, the time series data collected from the target user by a wearable sensor;
encoding the day-specific time series data;
performing a temporal pattern aggregation on the encoded day-specific time series data; and
performing Siamese-triplet network optimization on the aggregated data; and
determining an inference from the time series data based upon the analysis.

2. The method of claim 1, wherein the method further comprises performing a binary-class classification task.

3. The method of claim 1, wherein the method further comprises performing a multi-class classification task.

4. The method of claim 1, wherein the method further comprises determining whether the day-specific time series data for a target user represents a known user.

5. The method of claim 1, wherein the method further comprises determining a demographic class for the target user.

6. The method of claim 1, wherein the method further comprises determining a job performance prediction for the target user.

7. The method of claim 1, wherein the wearable sensor is an electronic heart rate monitor.

8. A computer system, comprising:
at least one processor; and
one or more computer-readable media having stored thereon computer-executable instructions that are executable by the at least one processor to cause the computer system to analyze time series data from a wearable sensor, the computer-executable instructions including instructions that are executable by the at least one processor to cause the computer system to perform at least:
performing an analysis of time series data by:
accessing day-specific time series data for a target user, the time series data collected from the target user by a wearable sensor;
encoding the day-specific time series data;
performing a temporal pattern aggregation on the encoded day-specific time series data; and
performing Siamese-triplet network optimization on the aggregated data; and
determining an inference from the time series data based upon the analysis.

9. The system of claim 8, wherein the system is further configured to perform a binary-class classification task.

10. The system of claim 8, wherein the system is further configured to perform a multi-class classification task.

11. The system of claim 8, wherein the system is further configured to perform determining whether the day-specific time series data for a target user represents a known user.

12. The system of claim 8, wherein the system is further configured to perform determining a demographic class for the target user.

13. The system of claim 8, wherein the system is further configured to perform determining a job performance prediction for the target user.

14. The system of claim 8, wherein the wearable sensor is an electronic heart rate monitor.

15. A computer program product comprising one or more hardware storage devices having stored thereon computer-executable instructions that are executable by at least one processor to cause a computer system to analyze time series data from a wearable sensor, the computer-executable instructions including instructions that are executable by the at least one processor to cause the computer system to at least perform:
performing an analysis of time series data by:
accessing day-specific time series data for a target user, the time series data collected from the target user by a wearable sensor;
encoding the day-specific time series data;
performing a temporal pattern aggregation on the encoded day-specific time series data; and
performing Siamese-triplet network optimization on the aggregated data; and
determining an inference from the time series data based upon the analysis.

16. The computer program product of claim 15, wherein the instructions are further configured to to cause the computer system to perform a multi-class classification task.

17. The computer program product of claim 15, wherein the instructions are further configured to to cause the computer system to to perform determining whether the day-specific time series data for a target user represents a known user.

18. The computer program product of claim 15, wherein the instructions are further configured to to cause the computer system to perform determining a demographic class for the target user.

19. The computer program product of claim 15, wherein the instructions are further configured to cause the computer system to perform determining a job performance prediction for the target user.

20. The computer program product of claim 15, wherein the wearable sensor is an electronic heart rate monitor.
